(19) United States
(12) Patent Application Publication
(10) Pub. No.: US 2007/0104670 A1
(43) Pub. Date: May 10, 2007

(54) Cosmetic composition comprising at least one specific fixative polymer and at least one sulphonated polymer

(76) Inventors: Christine Annotel, Paris (FR); Ludivine Laurent, Courbevoie (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/589,072

(22) Filed: Oct. 30, 2006

Related U.S. Application Data
(60) Provisional application No. 60/761,390, filed on Jan. 24, 2006.

(30) Foreign Application Priority Data
Oct. 28, 2005 (FR) 05 11090

(51) Int. Cl. A61K 8/81

(52) U.S. Cl. 424/70.15; 424/70.16

(57) ABSTRACT

The present invention relates to a cosmetic composition for treating keratin fibers, especially human keratin fibers such as the hair, comprising in a cosmetically acceptable medium: at least one fixative polymer comprising as monomers at least a vinyl lactam, an ethylenically unsaturated carboxylic acid and an alkyl acrylate or methacrylate whose alkyl moiety contains at least 6 carbon atoms, and at least one partially or fully neutralized sulphonated polymer containing at least one unit derived from a sulpho-functional ethylenically unsaturated monomer.

This application claims benefit of U.S. Provisional Application No. 60/761,390 filed Jan. 24, 2006, the contents of which are incorporated herein by reference. This application also claims benefit of priority under 35 U.S.C. § 119 to French Patent Application No. FR 05 11090, filed Oct. 28, 2005, the contents of which are also incorporated herein by reference.

The present invention relates to a cosmetic composition for treating keratin fibers, for example, human keratin fibers such as the hair, which comprises at least one specific fixative polymer and at least one sulphonated polymer, to the use of this composition for shaping and/or holding the hairstyle, and to a cosmetic treatment method employing it.

In the field of styling, such as among hair products intended for shaping and/or holding the hairstyle, hair compositions are often composed of a solution, usually an alcoholic or aqueous solution, and of one or more fixative polymers in a blend with various cosmetic adjuvants.

These compositions may take the form of hair gels or hair mousses, which may be applied to wetted hair prior to a blow-dry or other drying operation.

Hair gels may be composed of one or more thickening polymers or gelling agents in combination with one or more fixative polymers, whose function is usually to form a film on the surface of the keratin fibers to be fixed, in order to produce points of bonding between these fibers.

These hair gels may have a sliding texture which allows them to spread easily over the body of the hair without leaving a sticky or retardive sensation on application.

Moreover, these hair gels may also have a melting texture, which allows them to be absorbed more readily at the surface of the hair.

However, the combination of fixative polymers with one or more thickening polymers or gelling agents in hair gels may give rise, with greater or lesser rapidity, to the formation of unattractive residues on the hair, resulting from the partial breakdown of the bonding points.

Consequently there is a real need to find cosmetic compositions, such as for styling, which make it possible to minimize the formation of unattractive residues on the hair while giving the hair a high level of fixation, in order to obtain satisfactory shaping and/or shape retention of the hairstyle.

Surprisingly and advantageously, the present inventors have now discovered that, by combining a specific fixative polymer and a sulphonated polymer containing at least one unit derived from a sulpho-functional ethylenically unsaturated monomer, it is possible to minimize the formation of aggregates on the hair and/or to obtain satisfactory cosmetic properties.

Moreover, a combination of this kind also makes it possible to obtain a styling gel which exhibits a satisfactory texture, in other words a melting and sliding texture.

The present disclosure accordingly provides a cosmetic composition for treating fibers, for example. human keratin fibers such as the hair, that comprises such a combination.

The present disclosure further provides for the use of the composition according to the disclosure for shaping and/or retaining the hairstyle.

The present disclosure likewise relates to an aerosol device comprising a cosmetic composition according to the disclosure.

Also provided is a cosmetic treatment method employing the composition according to the disclosure.

Other subject-matter, features, aspects and advantages of the invention will emerge more clearly from reading the description and examples which follow.

According to the present disclosure, the cosmetic composition for treating keratin fibers, for example, human keratin fibers such as the hair, comprises, in a cosmetically acceptable medium:

at least one anionic fixative polymer comprising as monomers at least a vinyl lactam, an ethylenically unsaturated carboxylic acid and an alkyl acrylate or methacrylate whose alkyl moiety contains at least 6 carbon atoms, and

at least one partially or fully neutralized sulphonated polymer containing at least one unit derived from a sulpho-functional ethylenically unsaturated monomer.

Anionic Fixative Polymers

As the term is used herein, a “fixative polymer” is any polymer which allows a shape to be given or allows a given shape or style to be retained.

In at least one embodiment, the ethylenically unsaturated carboxylic acid is chosen from acrylic acid, methacrylic acid, itaconic acid and crotonic acid. In a further embodiment, the ethylenically unsaturated carboxylic acid is chosen from acrylic and methacrylic acid, and in a still further embodiment, is acrylic acid.

The vinyl lactam is, in at least one embodiment, vinylpyrrolidone.

The alkyl acrylate or methacrylate containing at least 6 carbon atoms is, in at least one embodiment, an acrylic or methacrylic ester containing an alkyl radical containing 8 to 18 carbon atoms.

In at least one embodiment, the alkyl radicals are chosen from 2-ethylhexyl, octyl, lauryl and stearyl radicals.

These polymers may be optionally crosslinked.

The anionic fixative polymers which can be used in the composition according to the present disclosure may be prepared, for example, by the process described in U.S. Pat. No. 5,015,708.

In at least one embodiment, the anionic fixative polymers which can be used in the composition according to the present disclosure comprise as monomers vinylpyrrolidone, acrylic acid and lauryl methacrylate.

A polymer used in at least one embodiment is the polymer sold under the name Acrylidone LM by ISP, which is a terpolymer of vinylpyrrolidone (68% by weight)/acrylic acid (23% by weight) and lauryl methacrylate (9% by weight).

The at least one anionic fixative polymer based on vinyl lactam, ethylenically unsaturated carboxylic acid and alkyl acrylate or methacrylate is present in the composition, in at least one embodiment, at a concentration ranging from 0.05% to 30% by weight, such as a concentration ranging from 0.1% to 20% by weight, and further such as a concentration ranging from 0.5% to 10% by weight, relative to the total weight of the composition.

Sulphonated Polymers

A sulpho-functional ethylenically unsaturated monomer is, for the purposes of the present disclosure, a monomer which possesses at least one double bond and which carries in its structure a group SO₃X (wherein X is chosen from hydrogen, from ions of an alkali metal or alkaline earth metal, from ammonium ions and from ions originating from an organic amine).

The sulpho-functional ethylenically unsaturated monomers useful herein are chosen, in at least one embodiment, from vinylsulphonic acid, styrenesulphonic acid, (meth)acrylamido(C₁-C₂₂)alkylsulphonic acids, N-(C₁-C₂₂)alkyl(meth)acrylamido(C₁-C₂₂)alkylsulphonic acids such as undecyl-acrylamidomethane-sulphonic acid and also their partially or fully neutralized forms.

In a further embodiment, the ethylenically unsaturated monomers are chosen from (meth)acrylamido(C₁-C₂₂)alkylsulphonic acids such as, for example, acrylamidomethanesulphonic acid, acrylamidoethanesulphonic acid, acrylamidopropanesulphonic acid, 2-acrylamido-2-methylpropanesulphonic acid, methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid, 2-acrylamido-2,6-dimethyl-3-heptanesulphonic acid and also their partially or fully neutralized forms.

For example, 2-acrylamido-2-methylpropanesulphonic acid (AMPS), and also its partially or fully neutralized forms, can be used in at least one embodiment.

The sulphonated polymers according to the present disclosure may be crosslinked or non-crosslinked. In at least one embodiment, crosslinked sulphonated polymers are used.

The phrase “partially neutralized” is meant, for the purposes of the present disclosure, sulphonic groups in salt form which are present in the sulphonated polymer in a proportion of 1% to 99%.

In at least one embodiment, the sulphonated polymers in accordance with the present disclosure are partially or fully neutralized by an inorganic base (sodium hydroxide, potassium hydroxide, aqueous ammonia) or an organic base such as mono-, di- or triethanolamine, an aminomethylpropanediol, N-methylglucamine, basic amino acids such as arginine and lysine, and mixtures of these compounds.

In at least one embodiment, the sulphonated polymers are at least 90% neutralized, i.e., they contain at least 90% of sulphonic groups in salt form.

Thus, in at least one embodiment, the sulphonated polymer(s) are poly(2-acrylamido-2-methylpropanesulphonic acid) polymers, crosslinked and at least 90% neutralized.

The poly(2-acrylamido-2-methylpropanesulphonic acid) polymers crosslinked and at least 90% neutralized comprise:

(a) from 90% to 99.9% by weight of units of formula (I):

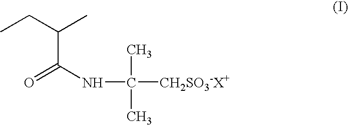

in which X⁺ is chosen from a proton, an alkali metal cation, an alkaline earth metal cation and an ammonium ion;

(b) from 0.01% to 10% by weight of crosslinking units originating from at least one monomer having at least two olefinic double bonds; the proportions by weight being defined in relation to the total weight of the polymer.

In at least one embodiment, the poly(2-acrylamido-2-methylpropanesulphonic acid) polymers crosslinked and at least 90% neutralized contain from 98% to 99.5% by weight of units of formula (I) and from 0.2% to 2% by weight of crosslinking units.

For example, in at least one embodiment, 90 to 100 mol % of the cations are NH₄⁺ cations and 0 to 10 mol % are protons.

The crosslinking monomers having at least two olefinic double bonds may be chosen, for example, from divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl(meth)acrylate, allyl ethers of alcohols from the sugar series, or other allyl or vinyl ethers of polyfunctional alcohols, and also the allyl esters of derivatives of phosphoric acid and/or vinylphosphonic acid, and mixtures of these compounds.

For example, use may be made of methylenebisacrylamide, allyl methacrylate or trimethylolpropane triacrylate (TMPTA).

The poly(2-acrylamido-2-methylpropanesulphonic acid) polymers crosslinked and at least 90% neutralized exhibit a viscosity as measured in a Brookfield viscometer using spindle 4 at a rotary speed of 100 revolutions/minute in 2% aqueous solution at 25° C. of greater than or equal to 10 poise (1000 cps).

In at least one embodiment, the crosslinked poly(2-acrylamido-2-methyl-propanesulphonic acid) polymers exhibit a viscosity as measured in a Brookfield viscometer using spindle 4 at a rotary speed of 100 revolutions/minute in 2% aqueous solution at 25° C. ranging from 50 to 400 poise (5000 to 40 000 cps), e.g., from 650 to 350 poise (6500 to 35 000 cps).

The sulphonated polymers which can be used in the cosmetic composition in accordance with the present disclosure may also be chosen from random amphiphilic AMPS polymers modified by reaction with a C₆-C₂₂ n-monoalkylamine or di-n-alkylamine, and of the kind described in patent application WO 00/31154, which is hereby incorporated by reference. These polymers may also contain, moreover, other units derived from ethylenically unsaturated hydrophilic monomers containing no fatty chain.

These ethylenically unsaturated monomers containing no fatty chain may be chosen, for example, from (meth)acrylic acids, their β-alkyl-substituted derivatives or their esters obtained with monoalcohols or with mono- or polyalkylene glycols, (meth)acrylamides, vinylpyrrolidone, maleic anhydride, itaconic acid or maleic acid, or mixtures of these compounds.

The sulphonated polymers may be chosen from amphiphilic copolymers of AMPS and at least one ethylenically unsaturated hydrophobic monomer containing at least one hydrophobic moiety having 6 to 50 carbon atoms, for example 6 to 22 carbon atoms, or 6 to 18 carbon atoms, or 10 to 18 carbon atoms and, in a still further embodiment, 12 to 18 carbon atoms.

These same copolymers may further contain at least one unit derived from ethylenically unsaturated monomers containing no fatty chain, such as (meth)acrylic acids, their β-alkyl-substituted derivatives or their esters obtained with monoalcohols or with mono- or poly-alkylene glycols, (meth)acrylamides, vinylpyrrolidone, maleic anhydride, itaconic acid or maleic acid, or mixtures of these compounds.

These copolymers are described in particular in patent application EP-A-750 899, in U.S. Pat. No. 5,089,578 and in the following publications by Yotaro Morishima:

Self-assembling amphiphilic polyelectrolytes and their nanostructures—Chinese Journal of Polymer Science Vol. 18, No. 40, (2000), 323-336.

Micelle formation of random copolymers of sodium 2-(acrylamido)-2-methylpropanesulfonate and a nonionic surfactant macromonomer in water as studied by fluorescence and dynamic light scattering—Macromolecules 2000, Vol. 33, No. 10-3694-3704;

Solution properties of micelle networks formed by non-ionic moieties covalently bound to a polyelectrolyte: salt effects on rheological behavior—Langmuir, 2000, Vol. 16, No. 12, 5324-5332;

Stimuli responsive amphiphilic copolymers of sodium 2-(acrylamido)-2-methylpropanesulfonate and associative macromonomers—Polym. Preprint, Div. Polym. Chem. 1999, 40(2), 220-221.

The ethylenically unsaturated hydrophobic monomers of these specific copolymers are chosen, in at least one embodiment, from acrylates or acrylamides of formula (II):

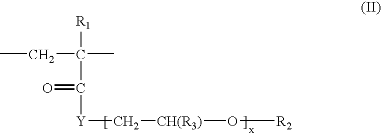

in which
R₁ and R₃, which are identical or different, are chosen from a hydrogen atom and linear or branched C₁-C₆ alkyl radicals, such as methyl;
Y is O or NH;
R₂ is a hydrophobic hydrocarbon radical comprising at least 6 to 50 carbon atoms, such as 6 to 22 carbon atoms, for example, 6 to 18 carbon atoms, or 10 to 18 carbon atoms and in a still further embodiment, 12 to 18 carbon atoms; and
x is the number of moles of alkylene oxide and ranges from 0 to 100.

The radical R₂ is chosen, in at least one embodiment, from linear C₆-C₁₈ alkyl radicals (for example n-hexyl, n-octyl, n-decyl, n-hexadecyl, n-dodecyl) and branched or cyclic C₆-C₁₈ alkyl radicals (for example cyclododecane (C₁₂) or adamantane (C₁₀)); perfluorinated alkyl C₆-C₁₈ radicals (for example the group of formula —(CH₂)₂—(CF₂)₉—CF₃); the cholesteryl radical (C₂₇) or a cholesterol ester residue such as the cholesteryl oxyhexanoate group; and aromatic polycyclic groups such as naphthalene or pyrene. In a further embodiment, R₂ is chosen from linear alkyl radicals, such as the n-dodecyl radical.

According to one embodiment of the present disclosure, the monomer of formula (II) comprises at least one alkylene oxide unit (x≧1) and is, for example, a polyoxyalkylenated chain. The polyoxyalkylenated chain, in at least one embodiment, is composed of ethylene oxide units and/or propylene oxide units and in a further embodiment, is composed of ethylene oxide units. The number of oxyalkylene units ranges from 3 to 100, such as from 3 to 50, and further, for instance, from 7 to 25.

Among these polymers mention may be made of the following:

crosslinked or non-crosslinked, neutralized or non-neutralized copolymers containing from 15% to 60% by weight of AMPS units and from 40% to 85% by weight of (C₈-C₁₆)alkyl(meth)acrylamide units or of (C₈-C₁₆)alkyl(meth)acrylate units relative to the polymer, such as those described in European Patent Application EP-A-750 899;

terpolymers containing from 10 to 90 mol % of acrylamide units, from 0.1 to 10 mol % of AMPS units and from 5 to 80 mol % of n-(C₆-C₁₈)alkylacrylamide units, such as those described in U.S. Pat. No. 5,089,578.

Mention may also be made of non-crosslinked copolymers of partially or fully neutralized AMPS and of dodecyl methacrylate, and also of non-crosslinked or crosslinked copolymers of partially or fully neutralized AMPS and of n-dodecylmethacrylamide, such as those described in the Morishima articles cited above.

In at least one embodiment, mention may be made of copolymers composed of 2-acrylamido-2-methylpropanesulphonic acid (AMPS) units of formula (I):

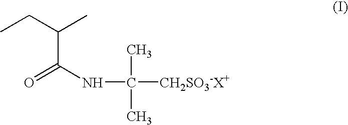

in which X⁺ is chosen from a proton, an alkali metal cation, an alkaline earth metal cation and an ammonium ion,

and of units of formula (III):

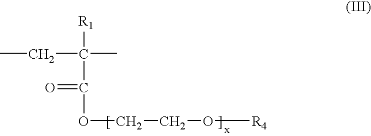

in which x is an integer ranging from 3 to 100, such as from 5 to 80, for instance from 7 to 25; R₁ has the same meaning as that indicated above in the formula (II), and R₄ is a linear or branched C₆-C₂₂. for example, C₁₀-C₂₂, alkyl radical.

The polymers used in at least one embodiment are those for which x=25, R₁ is methyl and R₄ is n-dodecyl; they are described in the abovementioned Morishima articles.

The polymers for which X⁺ is chosen from sodium and ammonium are used in at least one embodiment.

The percentage molar concentration of the units of formula (I) and of the units of formula (III) in the sulphonated polymers which can be used in the cosmetic composition according to the disclosure may range between 0.1 and 99.9 mol %.

For the more hydrophobic polymers the molar proportion of units of formula (II) or (III) may range from 50.1% to 99.9%, such as from 70% to 95%, and further, for example, from 80% to 90%.

For the less hydrophobic polymers, the molar proportion of units of formula (II) or (III) may range from 0.1% to 50%, such as from 5% to 25%, and further, for example, from 10% to 20%.

The sulphonated polymers which can be used in the cosmetic composition in accordance with the present disclosure may be obtained by conventional processes of free-radical polymerization in the presence of at least one initiator such as, for example, azobisisobutyronitrile (AIBN), azobisdimethylvaleronitrile, ABAH (2,2-azobis[2-amidino-propane]hydrochloride), organic peroxides such as dilauryl peroxide, benzoyl peroxide, tert-butyl hydroperoxide, etc., inorganic peroxide compounds such as potassium persulphate or ammonium persulphate, or H₂O₂, optionally in the presence of reductants.

The sulphonated polymers are obtained in a least one embodiment by free-radical polymerization in a tert-butanol medium, in which they precipitate.

Moreover, making use of polymerization by precipitation from tert-butanol, it is possible to obtain particles of the sulphonated polymer whose size distribution is beneficial to their use in at least one embodiment.

The size distribution of the particles of the polymer may be determined by example by laser diffraction or image analysis.

One distribution for this type of sulphonated polymer, as determined by image analysis, is as follows: 60.2% less than 423 microns, 52.0% less than 212 microns, 26.6% less than 106 microns, 2.6% less than 45 microns, and 26.6% greater than 850 microns.

The reaction may be conducted at a temperature ranging from 10 to 150° C., either at atmospheric pressure or under reduced pressure. It may also be carried out in an inert atmosphere, such as under nitrogen.

For example, the crosslinked poly(2-acrylamido-2-methylpropanesulphonic acid)s may be obtained by a preparation process comprising the following steps:

(a) the 2-acrylamido-2-methylpropanesulphonic acid monomer in free form is dispersed or dissolved in a solution of tert-butanol or of water and tert-butanol;

(b) the solution or dispersion of 2-acrylamido-2-methylpropanesulphonic acid monomer obtained in step (a) is neutralized by at least one organic or inorganic base, such as aqueous NH₃ ammonia, in an amount sufficient to give a degree of neutralization of the polymer's sulphonic acid functions ranging from 90% to 100%;

(c) the crosslinking monomer or monomers are added to the solution or dispersion in (b);

(d) a conventional free-radical polymerization is carried out in the presence of free-radical initiators at a temperature ranging from 10 to 150° C., the polymer precipitating in the solution or dispersion based on tert-butanol.

In at least one embodiment, the sulphonated polymer does not contain a hydrophobic chain.

The sulphonated polymers in accordance with the disclosure are present, in at least one embodiment, in an amount ranging from 0.05% to 20% by weight, such as from 0.1% to 10% by weight and further, for example, from 0.5% to 5% by weight, relative to the total weight of the hair treatment composition.

The weight ratio between the at least one anionic fixative polymer of the present disclosure and the at least one sulphonated polymer of the present disclosure is, in at least one embodiment, greater than 1.

Cosmetic Adjuvants

The cosmetic composition according to the disclosure may further comprise at least one cosmetic adjuvant chosen from fixative polymers other than those described above, dispersed, microdispersed or nanodispersed silicones in soluble form, non-polymeric thickeners, thickening polymers or gelling agents which are not sulphonated, cationic, anionic, amphoteric and nonionic surfactants, ester-type conditioning agents, antifoams, moisturizers, emollients, plasticizers, water-soluble and fat-soluble silicone or non-silicone sunscreens, permanent or temporary dyes, fragrances, peptizing agents, preservatives, ceramides and pseudoceramides, vitamins and provitamins including panthenol, proteins, sequestrants, solubilizers, alkalifiers, anticorrosion agents, fatty substances such as vegetable, animal, mineral and synthetic oils, reducing agents or antioxidants, and oxidizing agents.

The person skilled in the art will ensure that any adjuvants and their amount are selected in such a way as to cause no detriment to the properties of the compositions of the present disclosure.

The at least one cosmetic adjuvant is present, for example, at a concentration for each ranging from 0.001% to 50% by weight relative to the total weight of the composition.

Cosmetically Acceptable Medium

As used herein, by cosmetically acceptable medium is meant a medium which is compatible with the keratin materials, such as the hair.

The cosmetically acceptable medium may be an alcoholic, aqueous or aqueous-alcoholic medium. Thus the medium may, for example, comprise water only, alcohol only, or a mixture of water and at least one cosmetically acceptable solvent such as C₁-C₄ lower alcohols, polyols, polyol monoethers and mixtures thereof. In at least one embodiment, the C₁-C₄ lower alcohol is ethanol.

Packaging

The compositions in accordance with the present disclosure may be packaged in a tub, in a tube, in a pump flask or in an aerosol device which is customary in cosmetology.

The propellants used in the aerosol systems according to the present disclosure may be chosen from air, nitrogen, carbon dioxide, dimethyl ether, C₃ to C₅ alkanes, 1,1-difluoroethane and mixtures thereof.

The present disclosure likewise provides an aerosol device comprising the above-described composition and a means of distributing this composition.

Cosmetic Treatment and Use

The present disclosure also provides a cosmetic treatment method for the hair, for example a styling method, which comprises applying an effective amount of a composition described above to the wet or dry hair and in rinsing it off or leaving it on after a possible exposure time or after a possible drying operation.

The composition according to the disclosure is, in at least one embodiment, in the form of a gel which exhibits a viscosity of at least 2 poise at 25° C. (200 cps) using a Thermoelectron RS600 rheometer at a shear rate of 1 s⁻¹.

For example, the cosmetic composition exhibits a viscosity ranging from 2 to 1000 poise at 25° C. (200 and 100 000 cps at 25° C.), such as from 5 to 5000 at 25° C. (500 and 50 000 cps at 25° C.) and further, for example, from 8 to 3000 poise (800 and 30 000 cps at 25° C.) and a shear rate of 1 s⁻¹, as measurable using the RS600 rheometer from Thermoelectron.

The present disclosure likewise provides for the use of a cosmetic composition for shaping and/or retaining the hairstyle.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contain certain errors necessarily resulting from the standard deviation found in their respective testing measurements.

The example which follows is given by way of illustration and not of limitation of the present disclosure.

### EXAMPLE

A styling gel according to the present disclosure was prepared from the following compounds:

| | |
|---|---|
| vinylpyrrolidone/acrylic acid/lauryl methacrylate | 1% |
| terpolymer provided by ISP under the name | |
| ACRYLIDONE LM | |
| poly(2-acrylamido-2-methylpropanesulphonic acid) | 0.6% |
| crosslinked and neutralized with aqueous ammonia, | |
| provided by Clariant under the name | |
| HOSTACERIN AMPS | |
| preservatives | qs |
| water | qs 100% |

The percentages of each of the compounds in the styling gel were calculated by weight relative to the total weight of the composition.

A styling gel was obtained which had a viscosity of 15 poise at 25° C. (1500 cps at 25° C.) and a shear rate of 1 s⁻¹ and which exhibited after drying a sliding and melting texture which went easily onto the hair.

The hair was found, after drying, to have little if any residue on its surface.

What is claimed is:

1. A cosmetic composition for treating keratin fibers comprising, in a cosmetically acceptable medium,
at least one anionic fixative polymer comprising as monomers at least a vinyl lactam, an ethylenically unsaturated carboxylic acid and an alkyl acrylate or methacrylate whose alkyl moiety comprises at least 6 carbon atoms, and
at least one partially or fully neutralized sulphonated polymer comprising at least one unit derived from a sulpho-functional ethylenically unsaturated monomer.

2. A cosmetic composition according to claim 1, wherein the ethylenically unsaturated carboxylic acid is chosen from acrylic acid, methacrylic acid, itaconic acid and crotonic acid.

3. A cosmetic composition according to claim 2, wherein the ethylenically unsaturated carboxylic acid is acrylic acid.

4. A cosmetic composition according to claim 1, wherein the vinyl lactam is vinylpyrrolidone.

5. A cosmetic composition according to claim 1, wherein the alkyl acrylate or methacrylate comprising at least 6 carbon atoms is an acrylic or methacrylic ester comprising a C₁-C₁₈ alkyl radical.

6. A cosmetic composition according to claim 5, wherein the alkyl radicals of the acrylic or methacrylic esters are chosen from 2-ethylhexyl, octyl, lauryl and stearyl radicals.

7. A cosmetic composition according to claim 1, wherein the at least one anionic fixative polymer comprises as monomers vinylpyrrolidone, acrylic acid and lauryl methacrylate.

8. A cosmetic composition according to claim 1, wherein the at least one anionic fixative polymer is present in an amount ranging from 0.05% to 30% by weight relative to the total weight of the composition.

9. A cosmetic composition according to claim 8, wherein the at least one anionic fixative polymer is present in an amount ranging from 0.5% to 10% by weight, relative to the total weight of the composition.

10. A cosmetic composition according to claim 1, wherein the sulpho-functional ethylenically unsaturated monomer is chosen from vinylsulphonic acid, styrenesulphonic acid, (meth)acrylamido(C₁-C₂₂)alkylsulphonic acids, N-(C₁-C₂₂)alkyl(meth)acrylamido(C₁-C₂₂)alkylsulphonic acids and also their partially or fully neutralized forms.

11. A cosmetic composition according to claim 10, where the sulpho-functional ethylenically unsaturated monomer is chosen from acrylamidomethanesulphonic acid, acrylamidoethanesulphonic acid, acrylamidopropanesulphonic acid, 2-acrylamido-2-methylpropanesulphonic acid, methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid, 2-acrylamido-2,6-dimethyl-3-heptanesulphonic acid and also their partially or fully neutralized forms.

12. A cosmetic composition according to claim 11, wherein the sulpho-functional ethylenically unsaturated monomer is chosen from 2-acrylamido-2-methylpropanesulphonic acid (AMPS) and also its partially or fully neutralized forms.

13. A cosmetic composition according to claim 1, wherein the at least one sulphonated polymer is crosslinked.

14. A cosmetic composition according to claim 1, wherein the at least one sulphonated polymer is partially or fully neutralized by an organic or inorganic base.

15. A cosmetic composition according to claim 14, wherein the at least one sulphonated polymer is at least 90% neutralized.

16. A cosmetic composition according to claim 1, wherein the at least one sulphonated polymer is a poly(2-acrylamido-2-methylpropanesulphonic acid) crosslinked and at least 90% neutralized.

17. A cosmetic composition according to claim 16, wherein the poly(2-acrylamido-2-methylpropanesulphonic acid) crosslinked and at least 90% neutralized comprises, relative to the total weight of the polymer:
(a) from 90% to 99.9% by weight of units of formula (I): 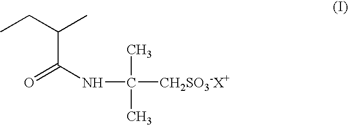
in which X⁺ is chosen from a proton, an alkali metal cation, an alkaline earth metal cation and an ammonium ion;
(b) from 0.01% to 10% by weight of crosslinking units originating from at least one monomer having at least two olefinic double bonds.

18. A cosmetic composition according to claim 17, wherein the crosslinked poly(2-acrylamido-2-methylpropanesulphonic acid) comprises from 98% to 99.5% by weight of units of formula (I) and from 0.2% to 2% by weight of crosslinking units.

19. A cosmetic composition according to claim 17, wherein in the formula (I) the cation X⁺ is NH₄⁺.

20. A cosmetic composition according to claim 16, wherein the poly(2-acrylamido-2-methylpropanesulphonic acid) is crosslinked by trimethylolpropane triacrylate.

21. A cosmetic composition according to claim 16, wherein the crosslinked poly(2-acrylamido-2-methylpropanesulphonic acid) exhibits a viscosity as measured in a Brookfield viscometer using spindle 4 at a rotary speed of 100 revolutions/minute in 2% aqueous solution at 25° C. of greater than or equal to 1000 cps.

22. A cosmetic composition according to claim 21, wherein the crosslinked poly(2-acrylamido-2-methylpropanesulphonic acid) exhibits a viscosity as measured in a Brookfield viscometer using spindle 4 at a rotary speed of 100 revolutions/minute in 2% aqueous solution at 25° C. ranging from 50 to 400 poise (5000 to 40 000 cps).

23. A cosmetic composition according to claim 21, wherein the crosslinked poly(2-acrylamido-2-methylpropanesulphonic acid) exhibits a viscosity as measured in a Brookfield viscometer using spindle 4 at a rotary speed of 100 revolutions/minute in 2% aqueous solution at 25° C. ranging from 65 to 350 poise (6500 to 35 000 cps).

24. A cosmetic composition according to claim 1, wherein the at least one sulphonated polymer further comprises at least one unit derived from an ethylenically unsaturated monomer containing no fatty chain.

25. A cosmetic composition according to claim 24, wherein the ethylenically unsaturated monomer containing no fatty chain is chosen from (meth)acrylic acids; β-alkyl-substituted derivatives of (meth)acrylic acids; esters of (meth)acrylic acids obtained with monoalcohols or with mono- or polyalkylene glycols; (meth)acrylamides; vinylpyrrolidine; maleic anhydride; itaconic acid; and maleic acid, and mixtures of these compounds.

26. A cosmetic composition according to claim 1, wherein the at least one sulphonated polymer is chosen from amphiphilic copolymers of AMPS and at least one ethylenically unsaturated hydrophobic monomer comprising at least one hydrophobic moiety having 6 to 50 carbon atoms.

27. A cosmetic composition according to claim 26, wherein the at least one hydrophobic moiety comprises 6 to 22 carbon atoms.

28. A cosmetic composition according to claim 27, wherein the at least one hydrophobic moiety comprises 12 to 18 carbon atoms.

29. A cosmetic composition according to claim 26, wherein the ethylenically unsaturated hydrophobic monomer is chosen from acrylates or acrylamides of formula (II): 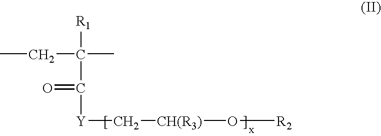
in which
R₁ and R₃, which are identical or different, are chosen from hydrogen and linear or branched C₁-C₆ alkyl radicals;
Y is O or NH;
R₂ is a hydrophobic hydrocarbon radical comprising at least 6 to 50 carbon atoms; and
x is the number of moles of alkylene oxide and ranges from 0 to 100.

30. A composition according to claim 29, wherein R₂ is a hydrophobic hydrocarbon radical comprising 12 to 18 carbon atoms.

31. A cosmetic composition according to claim 29, wherein the hydrophobic hydrocarbon radical R₂ is chosen from linear, branched or cyclic C₆-C₁₈ alkyl radicals; perfluorinated C₆-C₁₈ alkyl radicals; a cholesteryl radical; a cholesterol ester; and aromatic polycyclic groups.

32. A cosmetic composition according to claim 29, wherein the monomer of formula (II) further comprises at least one alkylene oxide unit (x≧1).

33. A cosmetic composition according to claim 29, wherein the monomer of formula (II) further comprises at least one polyoxyalkylenated chain.

34. A cosmetic composition according to claim 33, wherein the polyoxyalkylenated chain comprises ethylene oxide units and/or propylene oxide units.

35. A cosmetic composition according to claim 34, wherein the polyoxyalkylenated chain is composed solely of ethylene oxide units.

36. A cosmetic composition according to claim 29, wherein the number of oxyalkylene units ranges from 3 to 100.

37. A cosmetic composition according to claim 36, wherein the number of oxyalkylene units ranges from 7 to 25.

38. A cosmetic composition according to claim 26, wherein the sulphonated polymer is chosen from:
crosslinked or non-crosslinked copolymers comprising from 15% to 60% by weight of AMPS units and from 40% to 85% by weight of (C₈-C₁₆)alkyl(meth)acrylamide units or of (C₈-C₁₆)alkyl(meth)acrylate units, relative to the polymer;
terpolymers comprising from 10 to 90 mol % of acrylamide units, from 0.1 to 10 mol % of AMPS units and from 5 to 80 mol % of n-(C₆-C₁₈)alkylacrylamide units, relative to the polymer.

39. A cosmetic composition according to claim 26, wherein the at least one sulphonated polymer is chosen from:
non-crosslinked copolymers of partially or fully neutralized AMPS and n-dodecyl methacrylate, and
crosslinked or non-crosslinked copolymers of partially or fully neutralized AMPS and n-dodecylmethacrylamide.

40. A cosmetic composition according to claim 26, wherein the at least one sulphonated polymer is chosen from copolymers composed of 2-acrylamido-2-methylpropanesulphonic acid (AMPS) units of formula (I): 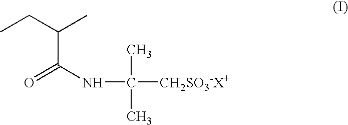
in which X⁺ is chosen from a proton, an alkali metal cation, an alkaline earth metal cation and an ammonium ion,
and of units of formula (III): 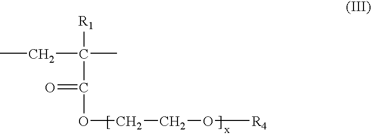
in which
x is an integer ranging from 3 to 100;
R₁ is chosen from hydrogen and linear or branched C₁-C₆ alkyl radicals; and
R₄ is a linear or branched C₆-C₂₂ alkyl radical.

41. A cosmetic composition according to claim 40, wherein x is an integer ranging from 7 to 25.

42. A cosmetic composition according to claim 40, wherein R₄ is a linear or branched C₁₀-C₂₂ alkyl radical

43. A cosmetic composition according to claim 40, wherein x is 25, R₁ is methyl and R₄ is n-dodecyl.

44. A cosmetic composition according to claim 29, wherein the units of formula (II) in the polymers are present in a percentage molar proportion ranging from 50.1% to 99.9%.

45. A cosmetic composition according to claim 40, wherein the units of formula (III) in the polymers are present in a percentage molar proportion ranging from 50.1% to 99.9%.

46. A cosmetic composition according to claim 44, wherein the percentage molar proportion of the units of formula (II) in the polymers ranges from 0.1% to 50%.

47. A cosmetic composition according to claim 45, wherein the percentage molar proportion of the units of formula (III) in the polymers ranges from 0.1% to 50%.

48. A cosmetic composition according to claim 1, wherein the at least one sulphonated polymer is present in a concentration ranging from 0.05% to 20% by weight, relative to the total weight of the composition.

49. A cosmetic composition according to claim 47, wherein the at least one sulphonated polymer is present at a concentration ranging from 0.5% to 5% by weight relative to the total weight of the composition.

50. A cosmetic composition according to claim 1, wherein said composition exhibits a viscosity ranging from 2 to 1000 poise at 25° C. (200 and 100 000 cps) and a shear rate of 1 s⁻¹.

51. A cosmetic composition according to claim 50, wherein said composition exhibits a viscosity ranging from 8 to 3000 poise at 25° C. (800 and 30 000 cps) and a shear rate of 1 s⁻¹.

52. A cosmetic composition according to claim 1, wherein the at least one anionic fixative polymer and the at least one sulphonated polymer are present in a weight ratio of greater than 1.

53. A cosmetic composition according to claim 1, further comprising at least one cosmetic adjuvant chosen from fixative polymers other than those disclosed in claim 1, dispersed, microdispersed or nanodispersed silicones in soluble form, non-polymeric thickeners, thickening polymers or gelling agents which are not sulphonated, cationic, anionic, amphoteric and nonionic surfactants, ester-type conditioning agents, antifoams, moisturizers, emollients, plasticizers, water-soluble and fat-soluble silicone or non-silicone sunscreens, permanent dyes, temporary dyes, fragrances, peptizing agents, preservatives, ceramides and pseudoceramides, vitamins and provitamins, proteins, sequestrants, solubilizers, alkalifiers, anticorrosion agents, fatty substances, reducing agents or antioxidants, and oxidizing agents.

54. A cosmetic composition according to claim 53, wherein the at least one cosmetic adjuvant is present at a concentration for each ranging from 0.001% to 50% by weight relative to the total weight of the composition.

55. A cosmetic composition according to claim 1, wherein the cosmetically acceptable medium is an aqueous, alcoholic or aqueous-alcoholic medium.

56. A cosmetic composition according to claim 55, wherein the aqueous-alcoholic medium comprises C₁-C₄ lower alcohols, polyols, polyol monoethers and mixtures thereof.

57. A cosmetic composition according to claim 56, wherein the C₁-C₄ lower alcohol is ethanol.

58. A cosmetic composition according to claim 1, wherein said composition is packaged in a vaporizer, a pump flask or an aerosol device.

59. A cosmetic composition according to claim 58, wherein said composition is packaged in an aerosol device.

60. A cosmetic composition according to claim 59, wherein said aerosol device comprises at least one propellant chosen from air, nitrogen, carbon dioxide, dimethyl ether, C₃ to C₅ alkanes, 1,1-difluoroethane and mixtures thereof.

61. An aerosol device formed by a container comprising a cosmetic composition and also a means of distributing the composition, wherein said composition comprises, in a cosmetically acceptable medium,
at least one anionic fixative polymer comprising as monomers at least a vinyl lactam, an ethylenically unsaturated carboxylic acid and an alkyl acrylate or methacrylate whose alkyl moiety comprises at least 6 carbon atoms, and
at least one partially or fully neutralized sulphonated polymer comprising at least one unit derived from a sulpho-functional ethylenically unsaturated monomer.

62. A method of cosmetic treatment for hair, comprising applying to the hair a cosmetic composition comprising, in a cosmetically acceptable medium,
at least one anionic fixative polymer comprising as monomers at least a vinyl lactam, an ethylenically unsaturated carboxylic acid and an alkyl acrylate or methacrylate whose alkyl moiety comprises at least 6 carbon atoms, and
at least one partially or fully neutralized sulphonated polymer comprising at least one unit derived from a sulpho-functional ethylenically unsaturated monomer, and
optionally rinsing said hair.

63. A cosmetic treatment method according to claim 62, wherein the application of said cosmetic composition is not followed by rinsing.

64. A method for shaping and/or retaining a hairstyle comprising applying to hair, in an amount effective to shape and/or retain a hairstyle, a cosmetic composition comprising, in a cosmetically acceptable medium,
at least one anionic fixative polymer comprising as monomers at least a vinyl lactam, an ethylenically unsaturated carboxylic acid and an alkyl acrylate or methacrylate whose alkyl moiety comprises at least 6 carbon atoms, and
at least one partially or fully neutralized sulphonated polymer comprising at least one unit derived from a sulpho-functional ethylenically unsaturated monomer.
